(19) United States
(12) Patent Application Publication
(10) Pub. No.: US 2014/0358163 A1
(43) Pub. Date: Dec. 4, 2014

(54) LAPAROSCOPIC GRASPERS

(71) Applicants: Danny Farin, Hod-Hasharon (IL); Yehuda Bachar, Givat-Shmuel (IL)

(72) Inventors: Danny Farin, Hod-Hasharon (IL); Yehuda Bachar, Givat-Shmuel (IL)

(73) Assignee: EON SURGICAL LTD., Tel-Aviv (IL)

(21) Appl. No.: 14/232,078

(22) PCT Filed: Jul. 11, 2012
(86) PCT No.: PCT/EP2012/063618

§ 371 Date: Aug. 20, 2014

Related U.S. Application Data
(60) Provisional application No. 61/506,595, filed on Jul. 11, 2011.

(51) Int. Cl. A61B 17/29; A61B 17/34

(52) U.S. Cl. CPC A61B 17/2909; A61B 17/3478; A61B 2017/2925; A61B 2017/2917; A61B 2017/2931; A61B 2017/00473
USPC 606/130

(57) ABSTRACT

An actuator for controlling a compressive force of a detachable laparoscopic grasper head. The actuator includes a handle unit comprising a movable lever, an outer tube member proximally connected to the handle unit and, when in use, distally connected to a first member of a detachable grasper head, a transmission member which comprises at least partially an elastic portion. The transmission member is axially arranged inside said outer tube member. The transmission member is proximally connected to the movable lever and, when in use, distally connected to a second member of the detachable grasper head. The transmission member is configured to transfer a lever force applied when moving the movable lever for actuating a compressive force by the grasping head depending on the lever force.

### FIELD AND BACKGROUND OF THE INVENTION

The present invention, in some embodiments thereof; relates to laparoscopic instruments and in particular to isometric laparoscopic graspers.

Surgical graspers are used for holding and manipulating bodily tissues during surgical procedures. Laparoscopic instruments, such as laparoscopic graspers, are frequently used in surgeries performed in abdominal cavity and are deployed through small punctures or incisions made in abdominal wall. In recent years, micro-laparoscopic surgical instruments were introduced that are characterized by significantly thinner shaft, connected to a surgical head, usually 3 mm or less in diameter. Special types of micro-laparoscopic instruments include interchangeable surgical heads that are detachably connectable to the thin shafts, optionally connected in the abdominal cavity and/or after shaft percutaneous introduction thereto. Main advantage in using interchangeable surgical heads lies in the possibility to use regular or increased sized heads which are more robust for surgical use but are substantially greater in diameter than the thin shaft. Such larger surgical heads are introducible to the abdominal cavity via regular sized laparoscopic ports (preferably a single port) and/or natural orifices and endoluminal passages.

Nevertheless, when using regular or large sized grasper head with a thin manipulator shaft thought has to be made to lessen any compromise in effectively delivering force from the handle to the head (to operate the grasper jaws to grasp with a chosen magnitude). Force is commonly delivered from an actuator (a lever or a handle in case of a manually operated grasper, or a motor in case of a machine or robotic operated grasper) through the shaft by relative displacement of the shaft members, usually an inner rod and an outer sheath capable of axially sliding in a range of relative positions. The inner rod and outer sheath are commonly connected distinctly to different members of the grasper head such that by relative axial displacement therebetween, the jaws will shift accordingly away or towards each other. Since that inner rod and outer sheath are minimized in cross section in affects design constraints on the ability to transfer large magnitude forces therethrough, as well as the ability to control the force in high accuracy.

Another consideration relates to grasper utilization at assembly. In order that the grasper will be effectively functional, the relative jaws juxtaposition should be fixedly correlated with relative axial positioning of the inner-rod/outer-sheath combination. These and other considerations are answered hereinafter.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing devices and methods according to the appended patent claims.

According to a first aspect, an actuator is provided for controlling a compressive force of a detachable laparoscopic grasper head. The actuator includes a handle unit comprising a movable lever. The actuator also includes an outer tube member proximally connected to the handle unit and, when in use, distally connected to a first member of a detachable grasper head. In some embodiments, the actuator includes a transmission member which comprises at least partially an elastic portion. The transmission member is optionally axially arranged inside the outer tube member. In some such embodiments, the transmission member is proximally connected to the movable lever and, when in use, distally connected to a second member of the detachable grasper head. Optionally, the transmission member is configured to transfer a lever force applied when moving the movable lever for actuating a compressive force by the grasping head depending on the lever force.

In some embodiments the elastic portion is axial stretchable and/or compressible. In some embodiments the elastic portion is the entire length of the transmission member.

In some embodiments the elastic portion includes variable elasticity along its length.

In some embodiments the elastic portion is an elastic transition portion interconnecting two substantially non-elastic parts of the transmission member.

In some embodiments the elastic transition portion is an extension spring having a free length and a maximal preset length when extended.

In some embodiments the extension spring is configured to stretch substantially proportionally to a spring constant above a threshold extension force applied thereto.

In some embodiments the extension spring is substantially rigid or non-elastic along its entire length below the threshold.

In some embodiments the elastic section is axially stretchable and/or compressible after a threshold extension force is applied to the elastic section.

In some embodiments the elastic section is substantially axially rigid or non-elastic below the threshold extension force.

In some embodiments the dependence of said compressive force on the lever force is substantially proportionally linear and having a first inclination, at least up to a predefined threshold.

In some embodiments the dependence has a second inclination when the threshold is exceeded, being substantially less steep than the first inclination.

In some embodiments the dependence at said second inclination is substantially proportional to an elongation of said elastic portion.

According to a second aspect, a method is provided for controlling a compressive force of a detachable grasping head attached to a manipulating shaft. The method includes providing a transmission member which comprises at least partially an elastic portion, axially inside an outer tube member, and proximally connected to a movable lever and distally connected to a second member of the detachable grasper head. Via the transmission member a lever force is transferred by moving the movable lever, thus actuating the grasping head with a compressive force, which is depending on the transferred lever force.

Some embodiments includes, stretching and/or compressing the elastic portion when a predetermine threshold force is exceeded and applied thereto.

Some embodiments includes, an applied lever force providing a compressive force depending on said stretching and/or compressing of the elastic portion.

Some embodiments includes, converting the isometric force to a proportional isotonic force after applying the threshold force to the elastic portion.

Some embodiments includes, converting the isometric force to a proportional regulated force after applying the threshold force to the elastic portion.

In a further aspect of the disclosure, an actuator is provided for setting a chosen relative positioning between movable members thereof, to corresponding movable members of a detachably connected laparoscopic grasper head, thereby defining chosen characteristics for controlling a compressive force by the detachable laparoscopic grasper head. The actuator includes a handle unit comprising at least one movable lever and a positioning means connected thereto. The actuator further includes a manipulator shaft which includes an outer tube member is connected at its proximal end to the handle unit and comprises a first connector at its distal end for connecting a first member of the detachable grasper head. The manipulator shaft also includes a slidable transmission member, which may include a slidable rod, axially arranged in a lumen of the outer tube member and connected at a proximal end to the movable lever. The slidable transmission member includes a second connector at its distal end for connecting a second member of the detachable grasper head. An applied force to the movable lever will move the first connector and the second connector relative each other. The positioning means is configured to allow immediate and/or automatic positioning the movable lever in a predetermined position, optionally imposing a chosen distance between the first connector and the second connector in absence of an actuating action on the handle.

In some advantageous outcomes according to this disclosure, the connectors will be positioned in chosen relation to each other, such that a surgical head is correctly connected and/or detached thereto in a way that facilitates its proper and/or most efficient utilization. The positioning of the movable lever by the positioning means may take place automatically when no force is applied to the movable lever by the practitioner or operator, for example if the positioning means includes a spring element.

Optionally, additionally or alternatively, means are provided in the actuator to improve force and/or haptic feedback. Optionally, such means increases the resistance in the movement of the movable lever in proportion to the lever distance from predetermined position as set by the positioning means. In some embodiments, the same positioning means may also be used for improving force and/or haptic feedback to the operator. The operator will thereby be aware that compression forces are applied by the grasper head. This will help the operator from over grasping and not apply excessive compression forces on the tissue being compressed by the grasping head.

The positioning means may be an active positioning means and as such may include at least one of a piston or a spring. The positioning means may alternatively be a passive positioning means and as such may include at least one of a stopper or a metering element.

In some embodiments the detachable grasper head is detachably connected such that opposing jaws thereof meet at a minimal distance imposed by the movable lever.

In some embodiments the movable lever, at and/or over the predetermined position, is adapted to transfer a variable force applied thereto as a compression force exercisable by the opposing jaws to a bodily tissue.

In some embodiments the positioning means is a spring element and/or a coiled spring and/or a piston and/or a stopper.

In some embodiments the positioning means provides a resist in a movement of the movable lever by extending and/or compressing the positioning element. In some embodiments the manipulator shaft includes a distal penetrating portion ending with a sharp distal tip capable of percutaneous insertion through bodily tissue layers.

In some embodiments the detachable grasper head is adapted to connect simultaneously to the first connector and the second connector at the predetermined distance.

In some embodiments the movable lever is adapted to move over a center point thereof when forced over the predetermined position

According to a further aspect, a method is provided for controlling a compressive force of a detachable laparoscopic grasper head. The method includes providing a handle unit connected at a proximal end of a manipulator shaft. The handle unit includes at least one movable lever and a positioning means connected thereto. By applying a force to said movable lever a first and second connector at the distal end of the manipulator shaft will move relative each other to obtain a distance therebetween. The method further includes positioning the movable lever by the positioning means to a predetermined position. Thereby imposing the distance to a predetermined distance between the first connector and the second connector in absence of an actuating action on the handle.

Some embodiments includes, using the positioning means to hinder the movable lever moving to a position where the distance is smaller than the predetermined distance, when applying a force thereto.

Some embodiments includes, allowing the movable lever moving to a position where the distance is smaller than the predetermined distance, when applying a force thereto. Some embodiments includes, using the positioning means to resist lever movement when moving the movable lever from the predetermined distance to a smaller distance.

Some embodiments includes, using the positioning means to resist lever movement when moving the movable lever.

According to an aspect of some embodiments of the present invention there is provided a laparoscopic instrument, such as a grasper, for controllably manipulating a bodily tissue. In some embodiments, the laparoscopic grasper includes a handle comprising at least one positionable lever, such as a movable lever. In some embodiments, the laparoscopic grasper comprises a manipulator shaft connected at its proximal end to the handle and comprising a first connector movable relatively to the handle per lever positions. In some embodiments, the laparoscopic grasper comprises a head (e.g., a surgical head) comprising opposing jaws detachably connected to the first connector, wherein the opposing jaws are adapted to meet at a minimal distance imposed by a chosen lever position. In some embodiments, the lever, at and/or over the chosen position, is adapted to transfer a variable force applied thereto as a compression force exercisable by the opposing jaws to a bodily tissue when disposed therebetween.

In some embodiments, the manipulator shaft includes a second connector, and the first connector is movable relatively to the second connector. In some embodiments, the manipulator shaft includes slidable concentric shafts. In some embodiments, the manipulator shaft includes a distal penetrating portion ending with a sharp distal tip capable of percutaneous insertion through bodily tissue layers. In some embodiments, the distal penetrating portion is equal or less than 3 mm in diameter.

In some embodiments, the head is adapted to connect to the connectors at a predetermined lever position. In some embodiments, the head is adapted to connect simultaneously to the first and second connectors at a predetermined distance between the first and second connectors.

In some embodiments, the lever is adapted to move over its center point when forced over the chosen position.

In some embodiments, the laparoscopic grasper further comprises resistive means to the relative motions of the manipulator shaft, thereby facilitating or improving force feedback during manual operation.

According to another aspect of some embodiments of the present invention there is provided an actuator for actuating an interchangeable surgical head, the actuator comprising:

a handle comprising a positionable lever, such as a movable lever;

a manipulator shaft, comprising:
a sheath having a length, a proximal end connected to the handle, a distal end comprising a first connector, and a lumen provided therebetween; and
an inner rod slidably movable in the lumen per the lever positions and comprising a second connector at a distal end thereof.

a lever positioning means adapted to position the lever in a chosen position thereby imposing a chosen distance between the first and second connectors;

wherein the interchangeable surgical head is connectable to the first and second connectors at the chosen distance.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 schematically illustrates a laparoscopic grasper comprising a detachable grasping head, in accordance embodiments of the present invention;

FIGS. 2A-C schematically illustrate side cut views demonstrating exemplary operational modes of an exemplary isometric laparoscopic grasper, in accordance with embodiments of the present invention;

FIGS. 3A-D illustrate views of exemplary manipulator shaft of an interchangeable surgical head actuator, in accordance with embodiments of the present invention;

FIGS. 4A-D schematically illustrate side cut views of different grasping jaws, in accordance with some embodiments of the present invention;

FIGS. 5A-C schematically illustrate different positions of an exemplary laparoscopic grasper comprising a grasping force controller, in accordance with some embodiments of the present invention; and

FIG. 6 is a schematic graph showing changes of compression forces applied to a grasped tissue in relation to proximity between lever and handle of the exemplary laparoscopic grasper in FIGS. 5A-C, in accordance with some embodiments of the present invention.

### DESCRIPTION OF EMBODIMENTS OF THE INVENTION

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

The following preferred embodiments may be described in the context of exemplary laparoscopic surgical procedures for ease of description and understanding. However, the invention is not limited to the specifically described devices and methods, and may be adapted to various clinical applications without departing from the overall scope of the invention. For example, devices and related methods including concepts described herein may be used for other medical interventions such as but not limited to: NOTES interventions, endoluminal interventions, GI surgeries, heart surgeries and general minimally invasive procedures.

The present invention, in some embodiments thereof, relates to a laparoscopic instrument, such as a laparoscopically introduced grasper, incorporating selective and controllable compression abilities. The present invention, in some embodiments thereof; also relates to means to allow improved manipulation as well as transferability of forces and moments to the grasped tissue using a laparoscopic instrument that is characterized by a regular sized interchangeable head detachably connected to a thin shaft.

In some embodiments of the present invention, the laparoscopic instrument includes a surgical end-effector, such as a grasper head, that is detachably connectable as an interchangeable part to a manipulator shaft, manually or otherwise operable, such as by using a dedicated handle. The manipulator shaft may be a micro-laparoscopic needle type introducer characterized by a distal intrusive portion having a maximal diameter of 3 mm or less, although the present invention applies as well to larger laparoscopic manipulator shafts including maximal diameters of 5 mm or more, or any other higher or intermediate size.

In some embodiments, the grasper head of the present invention may be considered as “surgical pliers” in the sense that it allows transferability of substantial compressive forces in-between the grasping jaws even at or after full contact. Optionally, such compressive forces may be limited or altered using means provided in the grasper head and/or the manipulation shaft. In some embodiments, a lever, such as a movable lever, may be used to shift the jaws closer or away, and once in contact, to create or transfer compressive forces in a selective, a continuous and/or a controllable manner. In an exemplary manually operable design, the lever is placed in the handle unit. In some embodiments, the actuator, such as a laparoscopic grasper operator, may include resistive means which resist manual actuation, at least over a predefined magnitude, thereby improve control and accuracy of the manual manipulation. Optionally, the lever arrangement creates a mechanical advantage, allowing the force of the hand's grip to be amplified and/or focused on an object with precision. The possibility of creating a continuous controllable and/or amplified grasping force may be referred to a laparoscopic grasper that is defined as “isometric”, in the sense that the variable grasping force is applied with no change in the relative distance and/or position of the two grasper jaws. Nevertheless, the actuating lever may be allowed to continue its travel when the jaws are in contact, while the hand's grip applied force increases.

In some embodiments, the interchangeable grasping head is connectable to a connector positioned at a distal portion of the manipulator shaft. The manipulator shaft may include two concentric longitudinal elements that are slidable one with the other along their lengths. Such mechanism may include an inner rod, such as a transmission member or a slidable transmission member, positioned and slidable in a lumen of an outer sheath, such as an outer tube member. At least one of the inner rod and the sheath includes connecting means to the interchangeable head or to any subcomponent thereof In an exemplary embodiment, the inner rod includes a first distal connector and the sheath includes a second distal connector, and the first and second connectors are connectable to different subcomponents of the interchangeable head, in such a way that any relative motion between the two connectors will cause a change to the interchangeable head form, as in the relative position of the grasping jaws thereof In some embodiments, a lever position, at least along a part of a full travel thereof will determine the jaws relative position.

In some embodiments, the grasping head is provided to be readily connectable to the manipulator shaft at a specific relative position between the opposing jaws, optionally when the jaws are in contact. In some embodiments, proper functionality of the grasping head is applicable when the first and second connectors of the manipulator shaft are positioned in a certain distance one to the other at the time of connection to the grasper head. In some embodiments, such a certain distance is set by a certain lever position, optionally a chosen and/or a predetermined position. In some embodiments, lever positioning means, such as a spring element or a stopper, is used to maintain the movable lever at a chosen position such as the one needed for the proper connectivity with the grasper head. Alternatively and/or additionally, the lever positioning means may in some examples be a resisting means.

An aspect of some embodiments of the present invention relates to a laparoscopic grasper for controllably manipulating a bodily tissue, comprising:

(1) a handle unit comprising at least one positionable lever, such as a movable lever;

(2) a manipulator shaft connected at its proximal end to the handle unit and comprising a first connector movable relatively to the handle unit, per the movable lever positions; and

(3) a head comprising opposing jaws detachably connected to said first connector, wherein said opposing jaws are adapted to meet at a minimal distance imposed by a chosen lever position.

In some embodiments, the lever, at and/or over the chosen position, is adapted to transfer a variable force applied thereto as a compression force exercisable by the opposing jaws to a bodily tissue when disposed therebetween.

In some embodiments, the manipulator shaft comprising a second connector and the first connector is movable relatively to the second connector. In some embodiments, the manipulator shaft comprises slidable concentric shafts.

In some embodiments, the manipulator shaft comprises a sheath, such as an outer tube member, having a length, a proximal end connected to the handle, a distal end comprising a first connector, and a lumen provided therebetween. In some embodiments, the manipulator shaft further includes an inner rod, such as a transmission member or a slidable transmission member, slidably movable in the sheath lumen per the lever positions and comprising a second connector at a distal end thereof.

In some embodiments, the manipulator shaft includes a distal penetrating portion ending with a sharp distal tip capable of percutaneous insertion through bodily tissue layers. Optionally, the distal penetrating portion is equal or less than 3 mm in diameter.

In some embodiments, the grasper head is adapted to connect to a connector at a predetermined lever position and/or to connect simultaneously to the first and second connectors at a predetermined distance between them.

Another aspect of some embodiments of the present invention relates to an actuator for actuating an interchangeable surgical head, such as a grasper head, which comprises a lever positioning means adapted to position the movable lever in a chosen position thereby imposing a chosen distance between the first and second connectors.

In some embodiments, the lever is adapted to move over its center point when forced over said chosen position.

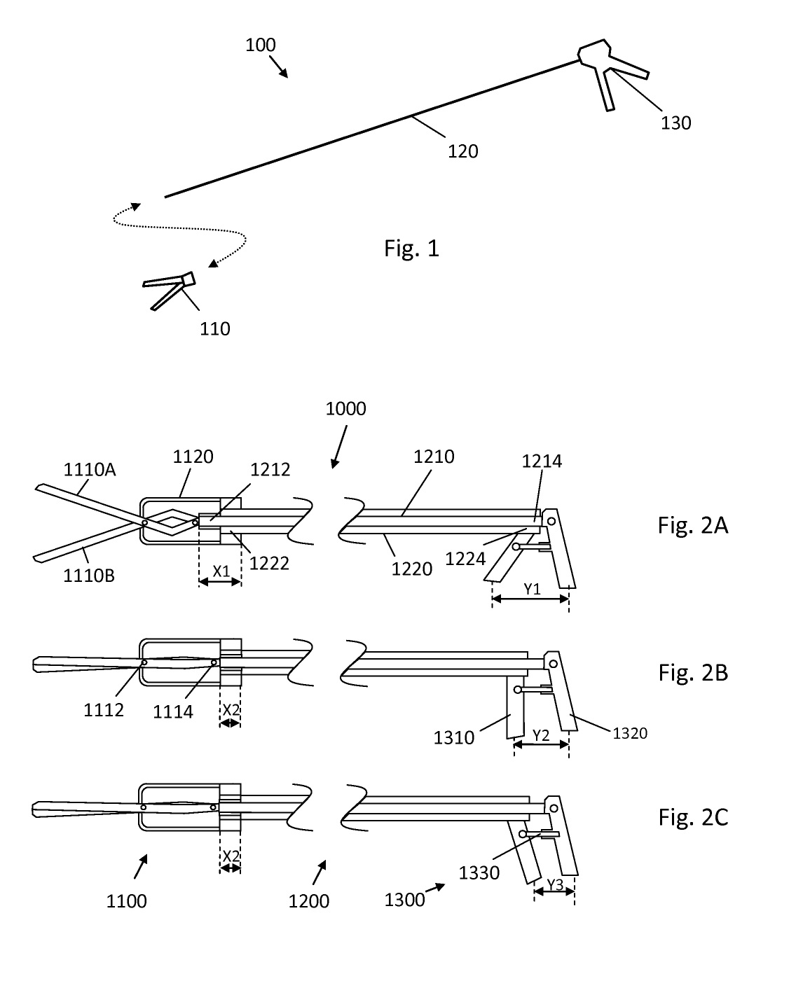

Referring now to the drawings, FIG. 1 schematically illustrates a laparoscopic grasper 100 comprising a detachable grasping head 110, in accordance embodiments of the present invention. Grasping head 110 is detachably connectable to manipulator shaft 120 which is readily connected at its proximal end to a handle 130. In some embodiments, manipulator shaft 120 is substantially thinner than grasping head 110 and may be equal or less than 3 mm in diameter, optionally equal or less than 2 mm, optionally approximately 1.5 mm. In some embodiments, handle 130 includes actuating means, such as a lever, that may transfer or create a variable grasping force in grasping head 110 via manipulator shaft 120, optionally a variable compression force that is fully controllable by the surgeon.

Reference is now made to FIGS. 2A-C which schematically illustrate side cut views demonstrating exemplary operational modes of an exemplary isometric laparoscopic grasper 1000, in accordance with embodiments of the present invention. Grasper 1000 includes a grasper head 1100, a manipulator shaft 1200 and a manually operable handle unit 1300. Grasping head 1100 includes two opposing jaws, an upper jaw 1110A and a lower jaw 1110B, interconnected as an exemplary embodiment as a 4-bar linkage mechanism, comprising a plurality of hinges or pins including a static hinge 1112 and an axially movable hinge 1114, and is at least partially housed in housing 1120. Accordingly, a relative distance and/or angle between the opposing jaws will be determined by the distance between movable hinge 1114 and static hinge 1112. In this particular example, a distancing of movable hinge 1114 from static hinge 1112 will bring jaws 1110A and 1110B closer (and vice versa), until a final (chosen, predetermined and/or preset according to manufacturer or user) distance is met in which the jaws are in direct contact by their inner surfaces at least partly along their length. In some embodiments, this direct contact blocks any further inward movement of the jaws. In some embodiments, grasper head 1100 and all subcomponents thereof are designed and configured to apply and maintain substantial compressive forces applicable by jaws 1110A and 1110B in-between, with or without an object (e.g., a bodily tissue) grasped therebetween. In some embodiments, compressive forces may optionally be equal to or above 0.5 N, optionally equal to or above 1 N, optionally equal to or above 5 N, optionally equal to or above 10 N, optionally equal to or above 50 N. Optionally, maximal compressive forces are kept below 30 N, optionally below 5 N, optionally between 0.1 to 2.5 N. In some embodiments, the allowed compressive pressure to a grasped tissue is 1 kPa, optionally 500 Pa, optionally 250 Pa.

Grasper head 1100 is detachably connected to manipulator shaft 1200 via a first connector 1212 located at a distal end of an inner rod, such as a transmission member or a slidable transmission member, 1210 and a second connector 1222 located at a distal end of a sheath, such as an outer tube member, 1220. Connectors 1212 and/or 1222 may include any type of connection means known to art, including bolting, threading, snap-locking, grasping and others. Optionally, both connectors 1212 and 1222 include threaded male components that can be threaded in corresponding threaded female components in grasper head 1100. In some embodiments, connector 1212 is directly linked with movable hinge 1114 and connector 1222 is directly linked with static hinge 1112, therefore any relative motion between the connectors will influence the distance between the hinges In some embodiments, inner rod 1210 is movable with relation to sheath 1220 in at least one axis, and in this particular example, inner rod 1210 can freely slide along the longitudinal axis of sheath 1220 lumen. Such sliding repositions relative distance of connectors 1212 and 1222 thereby influences jaws 1110A and 1110B distance and/or angle, and/or influences magnitude of compressive forces applied by the jaws once they are in contact.

Manipulator shaft 1200 is connected at its proximal end to handle unit 1300 in a configuration that allows subcomponents of handle unit 1300 to be used for operating grasper head 1100 via manipulator shaft 1200. In some embodiments, handle unit includes a static element and a moving element, such as a movable lever, or two moving elements, so that a relative repositioning between them will alter the distance between connectors 1212 and 1222. In this exemplary embodiment, handle unit 1300 includes a static handle 1320 hingedly connected to a movable lever 1310. In this particular example, handle 1320 is directly linked with a proximal end 1214 of inner rod 1210 and lever 1310 is directly linked with a proximal end 1224 of sheath 1220. Alternatively, as in many known devices, handle 1320 is directly linked with a proximal end 1224 of sheath 1220 and lever 1310 is directly linked with a proximal end 1214 of inner rod 1210. In some embodiments, lever 1310 operates grasper head 1100 according to its position or distance to handle 1320, and in this particular example, a manual extension of lever 1310 away from handle 1320 will cause sheath 1220 to be pulled proximally, thereby increasing the distance between connectors 1212 and 1222 thus opening jaws 1110A and 1110B, whereas a manually gripping of lever 1310 will cause the opposite reaction. FIG. 2A shows handle unit 1300 at maximal lever extension, referred to as distance Y1, in which movable hinge 1114 is at maximal distance X1 from proximal end of housing 1120 and jaws 1110A and 1110B are fully opened. FIG. 2B shows handle unit 1300 at a nominal, chosen and/or pre-set distance Y2 in which movable hinge 1114 is at minimal distance X2 from proximal end of housing 1120 and jaws 1110A and 1110B are fully closed.

In some embodiments, further manual gripping of lever 1310 at distance Y2 will increase the tension between inner rod 1210 and movable connector 1114 thereby causing jaws 1110A and 1110B to be forced one towards the other in a way that builds compression forces isometrically. Such isometric compression may be continuously controlled by the operator according to his hand's grip control. FIG. 2B shows a first handle unit 1300 design in which lever 1310 does not move closer under distance Y2 under increasing manual gripping. FIG. 2C shows a second design on handle unit 1300 where lever 1310 is allowed to move under manual gripping, for example up to a minimal distance Y3. In some embodiments, resisting means such as piston 1330 are provided in handle unit 1300 to resist lever movement between distance Y2 and Y3 for introducing or improving a force/haptic feedback to the operator. Additionally and/or alternatively the resisting mean may be positioning means. In such a way, the surgeon will be aware that compression forces are applied by grasper head 1100 and/or that the increasing resistance in correspondence to his increasing applied grasping will improve control and accurateness of the surgeon's operation. In some embodiments, the resisting means are provided as a spring element (e.g., a coiled spring) provided between inner rod 1210 and sheath 1220 (not shown), such as for example if provided around inner rod 1210 and connected in one end to inner rod 1210 and at its other end to sheath 1220. In some embodiments, relative motion between inner rod 1210 and sheath 1220, optionally above a predetermined force, will compress or extend the spring thereby increase manual sensitivity, manipulation accuracy and/or force feedback to the surgeon. In some embodiments, such feedback will take effect, or be mostly effective, only after jaws 1110A and 1110B are in contact.

Referring to the issue of connectivity between grasper head 1100 and manipulator shaft 1200, it should be noted that, at least in some designs related to the present invention, lever 1310 position at connection will affect grasping effectiveness and/or connection quality. As the distance between connectors 1212 and 1222 directly relates to the positions and displacement of jaws 1110A and 1110B, attention should be made to such variables in the instance of connection between grasper head 1100 and manipulator shaft 1200. This factor is especially important in scenarios where connection is made within patient's body (e.g., in the abdominal cavity) and/or when head grasper 1100 is held by delivery and/or encapsulating means (not shown) which allow certain formation of the jaws. In some embodiments, jaws 1110A and 1110B are kept closed (i.e., in contact along their inner surfaces) when grasper head 1100 is delivered and connected to manipulator shaft 1200, so that connectors 1212 and 1222 are positioned in advance in a certain distance which corresponds to the distance between hinges 1112 and 1114. In some embodiments, means are provided in handle unit 1300 to position lever 1310 at nominal distance Y2, at least when no substantial external forces are applied thereto, so that manipulator shaft 1200 will be ready for connection with grasper head 1100 when movable hinge 1114 is at minimal distance X2. In some embodiments, piston 1330 acts like a compression spring and/or includes or is provided with a spring (not shown) or other means to reposition lever 1310 at distance Y2 from any other position in the range between Y1 and Y3.

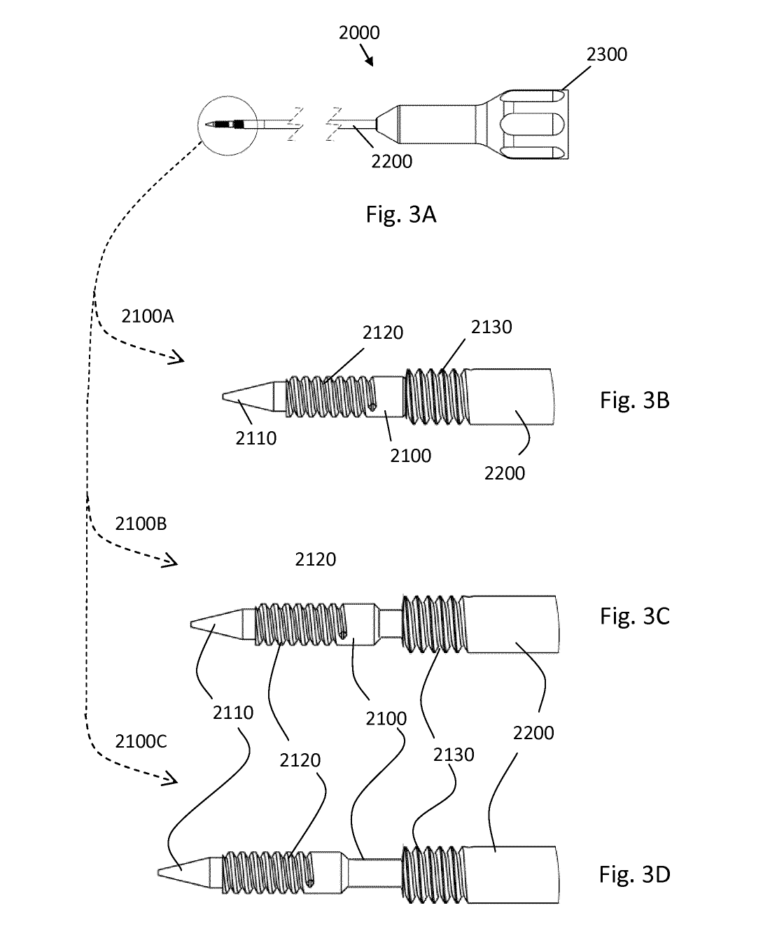

Reference is now made to FIGS. 3A-D which illustrate views of exemplary manipulator shaft 2000 of an interchangeable surgical head actuator, in accordance with embodiments of the present invention. Manipulator shaft 2000 includes a longitudinal sheath 2200 that is connected at its proximal end to nut-like connector 2300 which can be threaded over corresponding male thread of a handle unit (not shown). Manipulator shaft 2000 may be considered a micro-laparoscopy instrument with sheath 2200 having an outer diameter of 3 mm or less, and preferably 2 mm or less. Sheath 2200 may have a length of 50 cm or less, optionally not less than 20 cm.

An inner rod, such as a transmission member or a slidable transmission member, 2100 is in sheath, such as an outer tube member, 2200 lumen extending across its length and projecting out through its distal opening. In some embodiments, inner rod 2100 includes a sharp end 2110 so that manipulator shaft 2000 can be used to puncture into the patient's skin and penetrate through the dermal layers until reaching the abdominal cavity. Alternatively, inner rod 2100 may have a blunt tip in order to avoid harm to internal organs before connecting with a surgical head, so that auxiliary means are used in common practice to prepare a channel for manipulator shaft 2000 introduction and deployment. Inner rod 2100 is slidably movable in sheath 2200 in order to operate a surgical head connected thereto. Such a connection with a surgical head is made by its simultaneous screwing to a needle threading 2120 and sheath threading 2130 that are located concentrically and distant at a specific chosen distance.

As described above, in order that a grasper can function properly, the grasping head has to be assembled to the manipulator shaft such that a relative juxtaposition between the jaws of the grasper head will be fixedly correlated to relative axial positioning of inner rod and outer sheath of the manipulator shaft, at the instance of the assembly.

FIGS. 3B-D show enlarged views of a distal portion of manipulator shaft 2000, at three possible positions 2100A, 2100B and 2100C of inner rod 2100 with respect to sheath 2200. FIG. 3C shows an exemplary nominal position 2100B that is requested for a proper connection with a surgical head. In some embodiments, a grasper head according to the present invention is correctly connected to manipulator shaft 2000 at the nominal position 2100B shown in FIG. 3C having its jaws in full contact or partially open. In FIG. 3B, position 2100A is shown in which inner rod 2100 is withdrawn thereby causing a change in formation and/or actuation of a surgical head if in contact, optionally forcing jaws to optionally move one towards the other. In FIG. 3D, position 2100C is shown in which inner rod 2100 is further advanced distally thereby forcing jaws in a connected grasper head to optionally open. As previously described, in certain grasper heads designs, withdrawing inner rod 2100 will cause grasper jaws closing, and in other designs it may cause jaws opening, and vice versa.

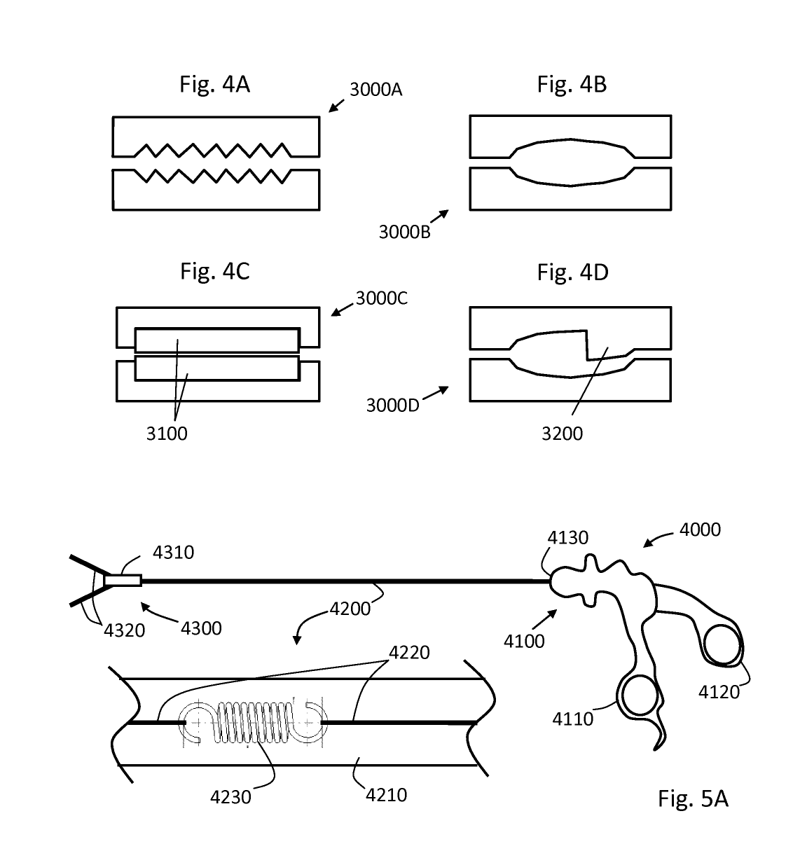

Special attention should be made also to the design of grasper head in order to facilitate effective isometric grasping or “pliers” functionality, including the design of the jaws. Reference is now made to FIGS. 4A-D which schematically illustrate side cut views of different grasping jaws, in accordance with some embodiments of the present invention. FIG. 4A illustrates exemplary jaws 3000A having teethed inner surfaces which can be useful for improving grasping and avoiding slipping especially of thin and/or smooth tissues. FIG. 4B illustrates exemplary “pliers” or “pincers” types jaws 3000B which allow compressive grasping of tissues at a distal portion while other portions of the grasped tissue are kept un compressed. FIG. 4C illustrates an exemplary jaws set 3000C which include cushioning means 3100 (optionally made of silicon rubber) to avoid harm to delicate tissues especially during compression and allow a more gradual compression thereto. FIG. 4D illustrates yet another type of jaws set 3000D comprising cutting means 3200 which can be useful for selectively cutting portions of a grasped tissue, similarly to the use of regular “pliers”.

As previously mentioned, once jaws are in direct contact or prevented from further approximation (e.g., by having a grasped object (e.g., tissue) compressed therebetween until developing a resistive force equal to the initially applied jaws-closing force), then by further increasing the manual squeezing force applied to the handle/lever, a proportional isometric force will develop. In cases of high manual forces, for example 1 kg or more, or in cases of very high manual forces, for example 5 kg or more, the applied isometric forces may harm the grasped tissue (e.g., being further compressed in a way that may irrecoverably deteriorate it) and/or to parts of the laparoscopic grasper (e.g., members or other parts that transfer the force from the handle/lever through the grasping head and to the tissue).

In some embodiments, a laparoscopic grasper includes means to control at least some aspects of the applied isometric force in a way that diminishes or prevents such harm. Optionally, such means limit the isometric force to a predetermined maximal force. Optionally, alternatively or additionally, such means decrease the isometric force to a smaller actual grasping force with a predetermined ratio, which may be constant or variable according to the force. Optionally, alternatively or additionally, such means may be set to replace a rigid transmission of the applied force to the grasping head to an elastic transmission, optionally over a predetermined value, therefore changing the isometric force to an isotonic force type (i.e., in which tension is constant while length changes) or to a regulated force type (i.e., in which tension changed while length changes in a predetermined ratio, different than the isometric ratio, and optionally up to a final predetermined value when maximal length is reached).

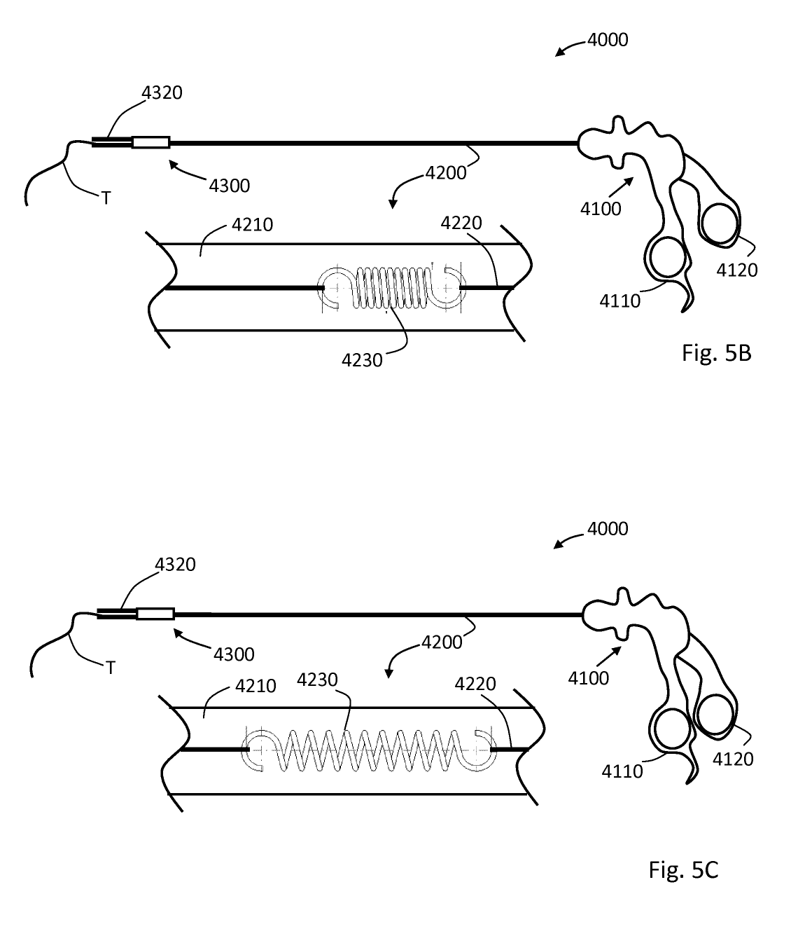

Reference is made to FIGS. 5A-C which schematically illustrate different positions of an exemplary laparoscopic grasper 4000 comprising a grasping force controller in the form of an elastic portion, such as an extension spring, 4230, in accordance with some embodiments of the present invention. Grasper 4000 includes a handle unit 4100, a grasper head 4300 and an elongated manipulator shaft 4200 interconnecting them and allowed transmission of power and motion from handle unit 4100 to grasper head 4300. Optionally, grasper head 4300 is detachably connectable to a distal end portion of manipulator shaft 4200. Optionally, handle unit 4100 is detachably connectable to a proximal end portion of manipulator shaft 4200. Handle unit 4100 includes a handle 4110 firmly connected (i.e., with no degree of freedom therebetween) to connector 4130 that is connected to manipulator shaft 4200. A movable lever 4120 is connected with handle 4110 by a hinge (not shown) in a way that allows fixed or variable pivoting thereabout.

Grasper head 4300 includes a base 4310 connected to manipulator shaft 4200 and allows transfer of power and/or motion from handle unit 4100 to jaws 4320 pivotally connected thereto. Jaws 4320 actuation is applicable by relative axial motion in manipulator shaft 4200 between an outer tube member 4210 proximally connected to handle 4110, and an enclosed rod member, such as a such as a transmission member or a slidable transmission member, 4220 proximally connected to lever 4120. Optionally, as previously described (yet not shown is FIG. 5), outer tube member 4210 and rod member 4220 both include connecting portions, concentrically disposed, each is detachably connected to different members in grasper head 4300 so that relative axial motion between them will actuate jaws 4320. In an exemplary embodiment, by pressing lever 4120 towards handle 4110, grasper head 4310 is actuated to close jaws 4320 together and/or increase compressive forces generated in-between.

In some embodiments, rod member 4220 is partly or fully elastic, or optionally includes elastic portions, or includes different parts or members, substantially rigid or otherwise substantially non-elastic, interconnected with at least one elastic portion. In this embodiment, the definition of “elastic” shall refer to being resistive to axial stretching and/or compressing and capable of substantially resuming original length after such stretching or compression. In an exemplary embodiment, rod member 4220 includes two parts interconnected with an elastic portion, such as an extension spring, 4230 having a free length (as shown in FIGS. 5A and 5B) and a maximal preset length (as shown in FIG. 5C). In some embodiments, spring 4230 is so chosen and/or prepared to begin stretching (i.e., allow initial substantial axial separation between at least two adjacent coils) only above a chosen threshold extension force axially applied thereto. Therefore, below such threshold, rod member 4220 as a whole shall function substantially as a rigid or otherwise non-elastic member along its entire length, so that only relative motion or no motion will take place between rod member 4220 and outer tube member 4210 without spring 4230 being stretched (while only, optionally, being axially displaced).

FIG. 5A shows grasper 4000 in a first position in which jaws 4320 are completely open and lever 4120 is maximally displaced from handle 4110.

FIG. 5B shows grasper 4000 in a second position in which jaws 4320 are closed upon a tissue T in a mild-to-no compression force applied thereto, and lever 4120 is partially displaced from handle 4110.

FIG. 5C shows grasper 4000 in a third position in which jaws 4320 are remained close upon tissue T but exert a predetermined maximal compression force thereto, and lever 4120 is in contact (i.e., zero distance) or at maximally allowed proximity, with handle 4110.

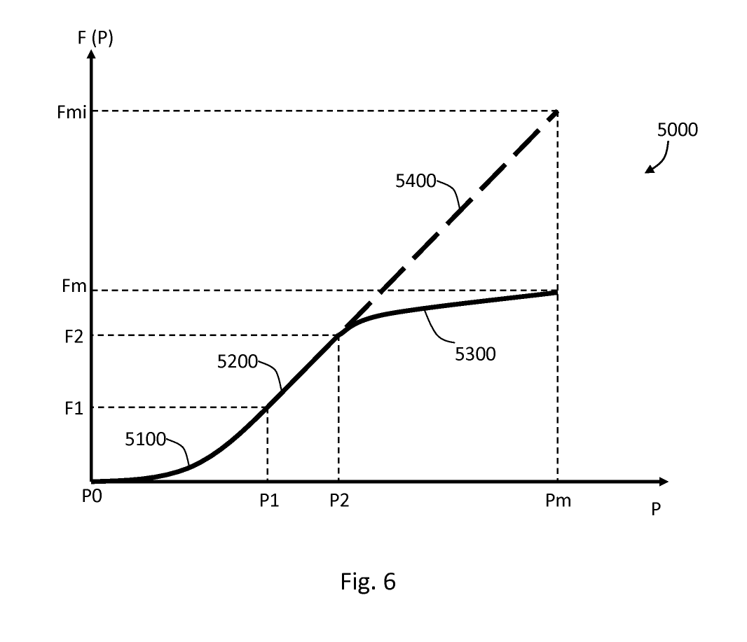

Reference is now made to FIG. 6, which is a schematic graph 5000 showing changes of compression forces F(P) applied to grasped tissue T in relation to proximity P between lever 4120 and handle 4110 of grasper 4000, in accordance with some embodiments of the present invention. Graph 5000 includes a first segment 5100 that schematically shows the change in compression force F(P) when lever 4120 is altered from an initial proximity position P0, in which lever 4120 is farthest from handle 4110 (as shown in FIG. 5A), to a first proximity position P1 in which jaws 4320 approach each other until closing and mildly compressing tissue T by force F1 (as shown in FIG. 5B). Tissue T is considered viscoelastic and therefore shown reacting with non-linear resistive force until reaching force F1, or sooner or later. Once exceeding force F1, the compressive force F(P) exerted by jaws 4320 may be considered isometric as jaws 4320 do not displaced while F(P) continue to change in accordance with continuously elevating manual force that forces lever 4120 to approximate handle 4110 until contact or until maximally allowed proximity.

In this exemplary embodiment, the extension force threshold for stretching spring 4230 is greater than F1 and equals to F2, tough in other possible embodiments, the threshold may be set to occur at or marginally to F1. Therefore, graph 5000 includes a second segment 5200 in which compression force F(P) changes from F1 to F2 while lever 4120 approximates from first proximity position P1 (as shown in FIG. 5B) to a second proximity position P2 (not shown). Along segment 5200, compression force F(P) is directly proportional to the manual force applied to handle unit 4100, therefore considered linear and incorporating a relatively steep inclination.

Upon exceeding F2, namely, spring extension threshold, spring 4230 elongates until reaching a maximal preset length shown in FIG. 5C. Segment 5300 shows the influence of spring 4230 expansion to the change in force F(P) between F2 and maximal compression force Fm, when lever 4120 is made in direct contact (as shown in FIG. 5C) with handle 4110 at maximal proximity position Pm. As shown, soon after stretching initiation (i.e., beginning of coils separation in spring 4230), graph segment 5300 regains linear proportion between F(P) and P though decrease in inclination angle in a manner that is mostly or fully dependant on spring 4230 constant.

Thus a lever force that is applied when moving said movable lever for actuating a compressive force by the grasping head is depending on said lever force.

In the example illustrated in FIG. 6, two linear portions can be seen. A first linear (substantially proportional) portion is present between F1 and F2. A second linear (substantially proportional) portion is present between F2 and Fm. The transition between the two portions might be slightly non linear adjacent the F2 threshold due to the start of stretching the elastic portion. The dependence of the grasping head's compressive force is thus providable as desired in dependence of the lever force applied. The ratio between the two forces is adjustable within the range of forces up to Fm, such as in the substantially proportional manner with a plurality of ranges with different inclination. This may be implemented by a plurality of elastic portions.

Alternatively, the second portion between F2 and Fm may be non-linear in specific examples. This may be implemented by choosing a desired spring constant behavior.

An illustrative graph segment 5400, showed in dashed line, represents the compression force F(P) inclination in the absent of a grasping force controller, in this exemplary embodiment, extension spring 4230. As shown, the maximal illustrative compression force Fmi in absence of spring 4230 is substantially greater than actual maximal compression force Fm, and may cause irrecoverable influence to tissue and/or grasper 4000 parts.

In some exemplary embodiments, force F1 is about 3N or smaller, optionally about 2N or smaller. Optionally, alternatively or additionally, force F2 is between about 1N and about 5N, optionally between about 2N and about 4N. Optionally, alternatively or additionally, force Fm is between about 3N and about 10N, optionally between about 4N and about 8N. Optionally, alternatively or additionally, illustrative force Fmi is about 10N or higher, optionally about 15N or higher, optionally about 20N or higher, optionally about 50N or higher, or any intermediate values.

Although the invention has been described in conjunction with specific embodiments thereof; it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An actuator for controlling a compressive force of a detachable laparoscopic grasper head, comprising:
a handle unit comprising a movable lever;
an outer tube member proximally connected to said handle unit and, when in use, distally connected to a first member of a detachable grasper head;
a transmission member which comprises at least partially an elastic portion, said transmission member is axially arranged inside said outer tube member, said transmission member is proximally connected to said movable lever and, when in use, distally connected to a second member of said detachable grasper head;
wherein said transmission member is configured to transfer a lever force applied when moving said movable lever for actuating a compressive force by said grasping head depending on said lever force.

2. The actuator according to claim 1, wherein said elastic portion is axially stretchable and/or compressible.

3. The actuator according to claim 1, wherein said elastic portion is the entire length of said transmission member.

4. The actuator according to claim 1, wherein said elastic portion includes variable elasticity along its length.

5. The actuator according to claim 1, wherein said elastic portion is an elastic transition portion interconnecting two substantially non-elastic parts of said transmission member.

6. The actuator according to claim 5, wherein said elastic transition portion is an extension spring having a free length and a maximal preset length when extended.

7. The actuator according to claim 6, wherein said extension spring is configured to stretch substantially proportionally to a spring constant above a threshold extension force applied thereto.

8. The actuator according to claim 7, wherein said extension spring is substantially non-stretchable below said threshold.

9-10. (canceled)

11. The actuator of claim 1, wherein said dependence of said compressive force on said lever force is substantially proportionally linear and having a first inclination, at least up to a predefined threshold.

12. The actuator of claim 11, wherein said dependence has a second inclination when said threshold is exceeded, being substantially less steep than said first inclination.

13. The actuator of claim 12, wherein said dependence at said second inclination is substantially proportional to an elongation of said elastic portion.

14-18. (canceled)

19. An actuator for controlling a compressive force of a detachable laparoscopic grasper head, the actuator comprising:
a handle unit comprising at least one movable lever and a positioning means connected thereto;
a manipulator shaft, comprising:
an outer tube member is connected at its proximal end to said handle unit and comprises a first connector at its distal end for connecting a first member of said detachable grasper head;
a slidable transmission member, such as a slidable rod, axially arranged in a lumen of said outer tube member and connected at a proximal end to said movable lever and, said slidable transmission member comprises a second connector at its distal end for connecting a second member of said detachable grasper head;
whereby an applied force to said movable lever will move said first connector and said second connector relative each other; and wherein said positioning means is configured to position said movable lever in a predetermined position thereby imposing a chosen distance between said first connector and said second connector in absence of an actuating action on said handle.

20. The actuator according to claim 19, wherein said detachable grasper head is detachably connected such that opposing jaws thereof meet at a minimal distance imposed by said movable lever.

21. The actuator according to claim 20, wherein said movable lever, at and/or over said predetermined position, is adapted to transfer a variable force applied thereto as a compression force exercisable by said opposing jaws to a bodily tissue.

22. The actuator according to claim 19, wherein said positioning means is a spring element and/or a coiled spring and/or a piston and/or a stopper.

23. The actuator according to claim 19, wherein said positioning means provides a resist in a movement of said movable lever by extending and/or compressing said positioning element.

24. The actuator according to claim 19, wherein said manipulator shaft includes a distal penetrating portion ending with a sharp distal tip capable of percutaneous insertion through bodily tissue layers.

25. The actuator according to claim 19, wherein said detachable grasper head is adapted to connect simultaneously to said first connector and said second connector at said predetermined distance.

26. The actuator according to claim 19, wherein said movable lever is adapted to move over a center point thereof when forced over said predetermined position.

27-31. (canceled)
